(19) United States
(12) Patent Application Publication
(10) Pub. No.: US 2003/0087843 A1
(43) Pub. Date: May 8, 2003

(54) O-pyrazole glucoside SGLT2 inhibitors and method of use

(76) Inventor: William N. Washburn, Titusville, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/235,336

(22) Filed: Sep. 5, 2002

Related U.S. Application Data
(60) Provisional application No. 60/317,280, filed on Sep. 5, 2001.

(51) Int. Cl. A61K 31/7052; C07H 17/02

(52) U.S. Cl. 514/43; 536/17.4

(57) ABSTRACT

A compound of formula I

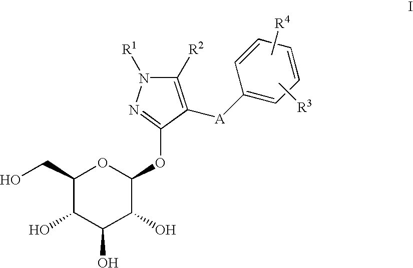

wherein

A is CH₂ or (CH₂)₂;

R¹ is hydrogen, arylalkyl, alkenyl, or alkyl;

R2 is alkyl or perfluoroalkyl; and

R³ and R⁴ are as defined herein.

Further provided are methods of using such compounds for the treatment of diabetes and related diseases, and to pharmaceutical compositions containing such compounds.

This application claims priority from U.S. Provisional Application No. 60/317,280 filed Sep. 5, 2001 which is incorporated herein by reference.

### FIELD OF THE INVENTION

The present invention relates to O-pyrazole glucosides which are inhibitors of sodium dependent glucose transporters found in the intestine and kidney (SGLT2) and to a method for treating diabetes, especially type II diabetes, as well as hyperglycemia, hyperinsulinemia, obesity, hypertriglyceridemia, Syndrome X, diabetic complications, atherosclerosis and related diseases, employing such O-pyrazole glucosides alone or in combination with one, two or more other type antidiabetic agent and/or one, two or more other type therapeutic agents such as hypolipidemic agents.

### BACKGROUND OF THE INVENTION

Approximately 100 million people worldwide suffer from type II diabetes (NIDDM), which is characterized by hyperglycemia due to excessive hepatic glucose production and peripheral insulin resistance, the root causes for which are as yet unknown. Hyperglycemia is considered to be the major risk factor for the development of diabetic complications, and is likely to contribute directly to the impairment of insulin secretion seen in advanced NIDDM. Normalization of plasma glucose in NIDDM patients would be predicted to improve insulin action, and to offset the development of diabetic complications. An inhibitor of the sodium-dependent glucose transporter SGLT2 in the kidney would be expected to aid in the normalization of plasma glucose levels, and perhaps body weight, by enhancing glucose excretion.

The development of novel, safe, and orally active antidiabetic agents is also desired in order to complement existing therapies, including the sulfonylureas, thiazolidinediones, metformin, and insulin, and to avoid the potential side effects associated with the use of these other agents.

Hyperglycemia is a hallmark of type II diabetes (NIDDM); consistent control of plasma glucose levels in diabetes can offset the development of diabetic complications and beta cell failure seen in advanced disease. Plasma glucose is normally filtered in the kidney in the glomerulus and actively reabsorbed in the proximal tubule. SGLT2 appears to be the major transporter responsible for the reuptake of glucose at this site. The SGLT2 specific inhibitor phlorizin or closely related analogs inhibit this reuptake process in diabetic rodents and dogs resulting in normalization of plasma glucose levels by promoting glucose excretion without hypoglycemic side effects. Long term (6 month) treatment of Zucker diabetic rats with an SGLT2 inhibitor has been reported to improve insulin response to glycemia, improve insulin sensitivity, and delay the onset of nephropathy and neuropathy in these animals, with no detectable pathology in the kidney and no electrolyte imbalance in plasma. Selective inhibition of SGLT2 in diabetic patients would be expected to normalize plasma glucose by enhancing the excretion of glucose in the urine, thereby improving insulin sensitivity, and delaying the development of diabetic complications.

Ninety percent of glucose reuptake in the kidney occurs in the epithelial cells of the early S1 segment of the renal cortical proximal tubule, and SGLT2 is likely to be the major transporter responsible for this reuptake. SGLT2 is a 672 amino acid protein containing 14 membrane-spanning segments that is predominantly expressed in the early S1 segment of the renal proximal tubules. The substrate specificity, sodium dependence, and localization of SGLT2 are consistent with the properties of the high capacity, low affinity, sodium-dependent glucose transporter previously characterized in human cortical kidney proximal tubules. In addition, hybrid depletion studies implicate SGLT2 as the predominant Na⁺/glucose cotransporter in the S1 segment of the proximal tubule, since virtually all Na-dependent glucose transport activity encoded in mRNA from rat kidney cortex is inhibited by an antisense oligonucleotide specific to rat SGLT2. SGLT2 is a candidate gene for some forms of familial glucosuria, a genetic abnormality in which renal glucose reabsorption is impaired to varying degrees. None of these syndromes investigated to date map to the SGLT2 locus on chromosome 16. However, the studies of highly homologous rodent SGLTs strongly implicate SGLT2 as the major renal sodium-dependent transporter of glucose and suggest that the glucosuria locus that has been mapped encodes an SGLT2 regulator. Inhibition of SGLT2 would be predicted to reduce plasma glucose levels via enhanced glucose excretion in diabetic patients.

SGLT1, another Na-dependent glucose cotransporter that is 60% identical to SGLT2 at the amino acid level, is expressed in the small intestine and in the more distal S3 segment of the renal proximal tubule. Despite their sequence similarities, human SGLT1 and SGLT2 are biochemically distinguishable. For SGLT1, the molar ratio of Na+to glucose transported is 2:1, whereas for SGLT2, the ratio is 1:1. The Kₘ for Na⁺ is 32 and 250-300 mM for SGLT1 and SGLT2, respectively. Kₘ values for uptake of glucose and the nonmetabolizable glucose analog α-methyl-D-glucopyranoside (AMG) are similar for SGLT1 and SGLT2, i.e. 0.8 and 1.6 mM (glucose) and 0.4 and 1.6 mM (AMG) for SGLT1 and SGLT2 transporters, respectively. However, the two transporters do vary in their substrate specificities for sugars such as galactose, which is a substrate for SGLT1 only.

Administration of phlorizin, a specific inhibitor of SGLT2 activity, provided proof of concept in vivo by promoting glucose excretion, lowering fasting and fed plasma glucose, and promoting glucose utilization without hypoglycemic side effects in several diabetic rodent models and in one canine diabetes model. No adverse effects on plasma ion balance, renal function or renal morphology have been observed as a consequence of phlorizin treatment for as long as two weeks. In addition, no hypoglycemic or other adverse effects have been observed when phlorizin is administered to normal animals, despite the presence of glycosuria. Administration of an inhibitor of renal SGLT2 for a 6-month period (Tanabe Seiyaku) was reported to improve fasting and fed plasma glucose, improve insulin secretion and utilization in obese NIDDM rat models, and offset the development of nephropathy and neuropathy in the absence of hypoglycemic or renal side effects.

Phlorizin itself is unattractive as an oral drug since it is a nonspecific SGLT1/SGLT2 inhibitor that is hydrolyzed in the gut to its aglycone phloretin, which is a potent inhibitor of facilitated glucose transport. Concurrent inhibition of facilitative glucose transporters (GLUTs) is undesirable since such inhibitors would be predicted to exacerbate peripheral insulin resistance as well as promote hypoglycemia in the CNS. Inhibition of SGLT1 could also have serious adverse consequences as is illustrated by the hereditary syndrome glucose/galactose malabsorption (GGM), in which mutations in the SGLT1 cotransporter result in impaired glucose uptake in the intestine, and life-threatening diarrhea and dehydration. The biochemical differences between SGLT2 and SGLT1, as well as the degree of sequence divergence between them, allow for identification of selective SGLT2 inhibitors.

The familial glycosuria syndromes are conditions in which intestinal glucose transport, and renal transport of other ions and amino acids, are normal. Familial glycosuria patients appear to develop normally, have normal plasma glucose levels, and appear to suffer no major health deficits as a consequence of their disorder, despite sometimes quite high (110 114 g/daily) levels of glucose excreted. The major symptoms evident in these patients include polyphagia, polyuria and polydipsia, and the kidneys appear to be normal in structure and function. Thus, from the evidence available thus far, defects in renal reuptake of glucose appear to have minimal long term negative consequences in otherwise normal individuals.

The following references disclose O-aryl glucosides as SGLT2 inhibitors for treating diabetes.

EP 598359A1 (also JP 035988) (Tanabe Seiyaku)discloses compounds of the following structure A:

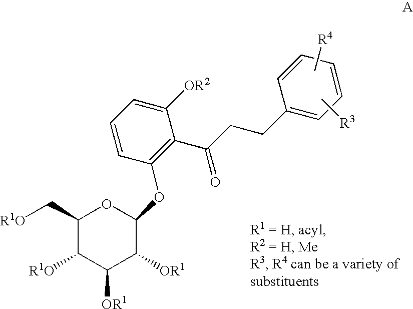

EP 0850948A1 discloses structures of the following genus B:

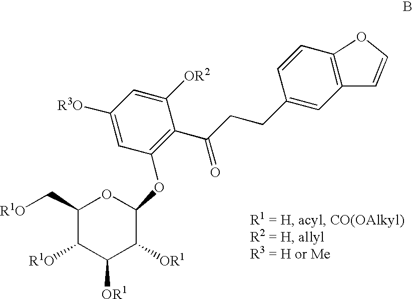

JP 09188625A expands upon structure B to include examples of B where R³ is H and where the 5 membered ring is saturated as well as the counterparts of benzothiophenes (X═S) and indenes (X═CH₂).

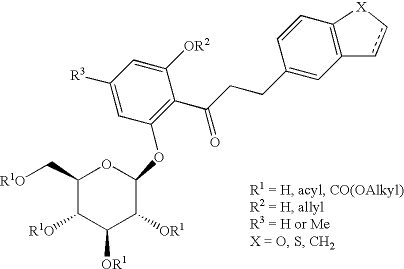

JP 09124685A expands upon structure B for R³═H to include derivatives of mono acylated C6 hydroxyl where the acyl group is a substituted benzoic or pyridyl carboxylic acid or a urethane generated from the corresponding phenol.

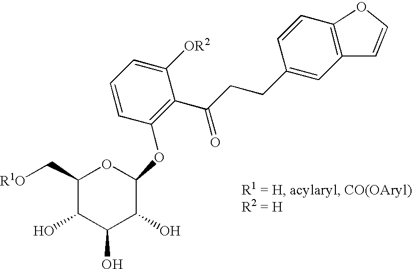

JP 09124684 discloses derivatives of structure B

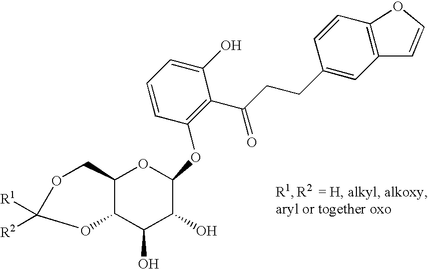

EP 773226-A1 discloses derivatives of structure B

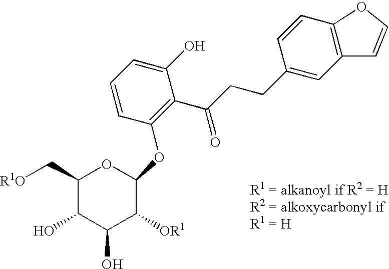

JP 08027006-A discloses derivatives of structure A where various combinations of the glucose hydroxyl are acylated and appears to be similar to EP 598359A1.

EP 0684254-A1 appears to encompass derivatives of structure B disclosed in JP 09188625A.

Other disclosures and publications which disclose SGLT2 inhibitors include the following:

K. Tsujihara et al, Chem. Pharm. Bull. 44, 1174-1180 (1996)

M. Hongu et al, Chem. Pharm. Bull. 46, 22-33 (1998)

M. Hongu et al, Chem. Pharm. Bull. 46, 1545-1555 (1998)

A. Oku et al, Diabetes, 48, 1794-1800 (1999)

JP 10245391 (Dainippon) discloses 500 structures as hypoglycemic agents for treatment of diabetes. These are O-glucosides of hydroxylated coumarins.

### SUMMARY OF THE INVENTION

In accordance with the illustrative embodiments and demonstrating features of the present invention, O-pyrazole glucoside compounds are provided which have the formula I.

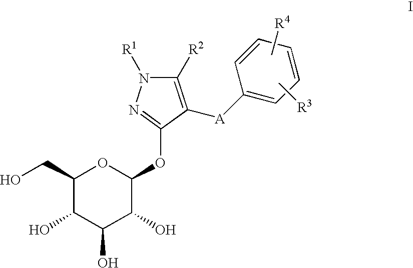

wherein

A is CH₂ or (CH₂)₂;

R¹ is hydrogen, arylalkyl, alkenyl, or alkyl;

R² is alkyl or perfluoroalkyl;

R³ and R⁴ are each independently hydrogen, OH, OR⁵, OAryl, OCH₂Aryl, alkyl, cycloalkyl, CF₃, —OCHF₂, -3,4-(OCH₂O), OCF₃, halogen, —CN, —CO₂R^{5a}, —CO₂H, —COR⁶, —CH(OH)R^{6a}, —CH(OR^{5b})R^{6b}, —CONR^{6c}R^{6d}, —NHCOR^{5c}, —NHSO₂R^{5d}, —NHSO₂Aryl, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂, or R³ and R⁴ together with the carbons to which they are attached form an annelated five, six or seven membered carbocycle or heterocycle which may contain 1 to 4 heteroatoms in the ring which are N, O, S, SO, and/or SO₂;

R⁵, R^{5a}, R^{5b}, R^{5c}, R^{5d}, R^{5e}, R^{5f}, and R^{5g}, are each independently alkyl; and

R⁶, R^{6a}, R^{6b}, R^{6c} and R^{6d} are each independently hydrogen, alkyl, aryl, arylalkyl or cycloalkyl, or R^{6c} and R^{6d} together with the nitrogen to which they are attached form an annelated five, six or seven membered heterocycle which may contain 1 to 4 heteroatoms in the ring which are N, O, S, SO, and/or SO₂.

The definition of formula I above inclues all pharmaceutically acceptable salts, stereoisomers, and prodrug esters of formula I.

The compounds of formula I possess activity as inhibitors of the sodium dependent glucose transporters found in the intestine and kidney of mammals and are useful in the treatment of diabetes and the micro- and macrovascular complications of diabetes such as retinopathy, neuropathy, nephropathy, and wound healing.

The present invention provides for compounds of formula I, pharmaceutical compositions employing such compounds and for methods of using such compounds. In particular, the present invention provides a pharmaceutical composition comprising a therapeutically effective amount of a compound of formula I, alone or in combination with a pharmaceutically acceptable carrier.

In addition, in accordance with the present invention, a method is provided for treating or delaying the progression or onset of diabetes, especially type I and type II diabetes, including complications of diabetes, including retinopathy, neuropathy, nephropathy and delayed wound healing, and related diseases such as insulin resistance (impaired glucose homeostasis), hyperglycemia, hyperinsulinemia, elevated blood levels of fatty acids or glycerol, obesity, hyperlipidemia including hypertriglyceridemia, Syndrome X, atherosclerosis and hypertension, and for increasing high density lipoprotein levels, wherein a therapeutically effective amount of a compound of formula I is administered to a mammalian, e.g., human, patient in need of treatment.

The compounds of the invention can be used alone, in combination with other compounds of the present invention, or in combination with one or more other agent(s) active in the therapeutic areas described herein.

In addition, a method is provided for treating diabetes and related diseases as defined above and hereinafter, wherein a therapeutically effective amount of a combination of a compound of formula I and at least one other type of therapeutic agent, such as an antidiabetic agent and/or a hypolipidemic agent, is administered to a human patient in need of treatment.

Preferred are compounds of formula I

wherein

A is CH₂;

R¹ is hydrogen or benzyl;

R³ and R⁴ are independently hydrogen, OR⁵, OAryl, OCH₂Aryl, -3,4-(OCH₂O), alkyl, cycloalkyl, CF₃, —OCHF₂, —OCF₃, halogen, —CO₂R^{5a}, —COR⁶, —CH(OH)R^{6a}, —CH(OR^{5b})R^{6b}, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂, or R³ and R⁴ together with the carbons to which they are attached form an annelated five, six or seven membered carbocycle.

Most preferred are compounds of formula I of the structure IA

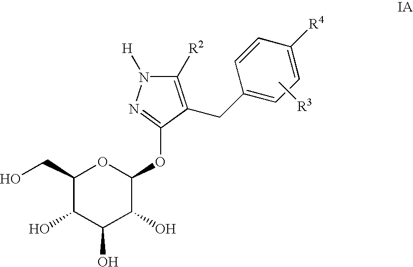

wherein

R³ is hydrogen; and

R⁴ is hydrogen, OR⁵, OAryl, OCH₂Aryl, -3,4-(OCH₂O), alkyl, cycloalkyl, CF₃, —OCHF₂, —OCF₃, halogen, —CO₂R^{5a}, —COR⁶, —CH(OH)R^{6a}, —CH(OR^{5b})R^{6b}, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂ and wherein R⁴ is positioned para to A.

### DETAILED DESCRIPTION OF THE INVENTION

The following abbreviations are employed herein:

Ac=acetyl

CHO=Chinese hamster ovary

Me=methyl

Et=ethyl

THF=tetrahydrofuran

EtOAc ethyl acetate

DMSO=dimethyl sulfoxide

DMF=dimethyl formamide

DME=dimethoxyethane

MeOH=methanol

HOAc or AcOH=acetic acid

min=minute(s)

h or hr=hour(s)

mL=milliliter

g=gram(s)

mg=milligram(s)

mol=mole(s)

mmol=millimole(s)

meq=milliequivalent

HPLC=high performance liquid chromatography

LC/MS=high performance liquid chromatography/mass spectrometry

NMR=nuclear magnetic resonance

M+H=parent plus a proton

YMC=trademark of YMC Co, Ltd., Kyoto, Japan

PBS=phosphate buffered saline

Ham's F-12=a cell growth medium commercially available from Life Technologies

The following definitions apply to the terms as used throughout this specification, unless otherwise limited in specific instances.

The term “lower alkyl”, “alkyl” or “alk” as employed herein alone or as part of another group includes both straight and branched chain hydrocarbons, containing 1 to 20 carbons, preferably 1 to 10 carbons, more preferably 1 to 8 carbons, in the normal chain, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, the various branched chain isomers thereof, and the like. Any of such groups may be optionally substituted with one or more substituents such as halo, for example F, Br, Cl or I or CF₃, alkyl, alkoxy, aryl, aryloxy, aryl(aryl) or diaryl, arylalkyl, arylalkyloxy,-alkenyl, alkynyl, cycloalkyl, cycloalkenyl, cycloalkylalkyl, cycloalkylalkyloxy, optionally substituted amino, hydroxy, hydroxyalkyl, acyl, oxo, alkanoyl, heteroaryl, heteroaryloxy, cycloheteroalkyl, arylheteroaryl, arylalkoxycarbonyl, heteroarylalkyl, heteroarylalkoxy, aryloxyalkyl, aryloxyaryl, alkylamido, alkanoylamino, arylcarbonylamino, nitro, cyano, thiol, haloalkyl, trihaloalkyl and/or alkylthio.

Unless otherwise indicated, the term “cycloalkyl” as employed herein alone or as part of another group includes saturated or partially unsaturated (containing 1 or more double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 10 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl and cyclododecyl, cyclohexenyl,

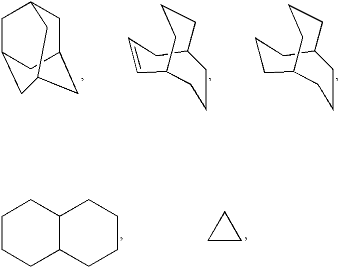

any of which groups may be optionally substituted with one or more substituents such as halogen, alkyl, alkoxy, hydroxy, aryl, aryloxy, arylalkyl, cycloalkyl, alkylamido, alkanoylamino, oxo, acyl, arylcarbonylamino, amino, nitro, cyano, thiol and/or alkylthio and/or any of the alkyl substituents.

Unless otherwise indicated, the term “alkenyl” or “lower alkenyl” as used herein by itself or as part of another group refers to straight or branched chain radicals of 2 to 20 carbons, preferably 2 to 12 carbons, and more preferably 1 to 8 carbons in the normal chain, which include one or more double bonds in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, and the like, and which may be optionally substituted with one or more substituents, namely, halogen, haloalkyl, alkyl, alkoxy, alkenyl, alkynyl, aryl, arylalkyl, cycloalkyl, amino, hydroxy, heteroaryl, cycloheteroalkyl, alkanoylamino, alkylamido, arylcarbonylamino, nitro, cyano, thiol, alkylthio and/or any of the alkyl substituents set out herein.

The terms “arylalkyl”, “arylalkenyl” and “arylalkynyl” as used alone or as part of another group refer to alkyl, alkenyl and alkynyl groups as described above having an aryl substituent. Representative examples of arylalkyl include, but are not limited to, benzyl, 2-phenylethyl, 3-phenylpropyl, phenethyl, benzhydryl and naphthylmethyl and the like.

Where alkyl groups as defined above have single bonds for attachment to other groups at two different carbon atoms, they are termed “alkylene” groups and may optionally be substituted as defined above for “alkyl”.

The term “halogen” or “halo” as used herein alone or as part of another group refers to chlorine, bromine, fluorine, and iodine, with chlorine or fluorine being preferred.

The term “metal ion” refers to alkali metal ions such as sodium, potassium or lithium and alkaline earth metal ions such as magnesium and calcium, as well as zinc and aluminum.

Unless otherwise indicated, the term “aryl” or “Aryl” as employed herein alone or as part of another group refers to monocyclic and bicyclic aromatic groups containing 6 to 10 carbons in the ring portion (such as phenyl or naphthyl including 1-naphthyl and 2-naphthyl) and may optionally include one to three additional rings fused to a carbocyclic ring or a heterocyclic ring (such as aryl, cycloalkyl, heteroaryl or cycloheteroalkyl rings for example

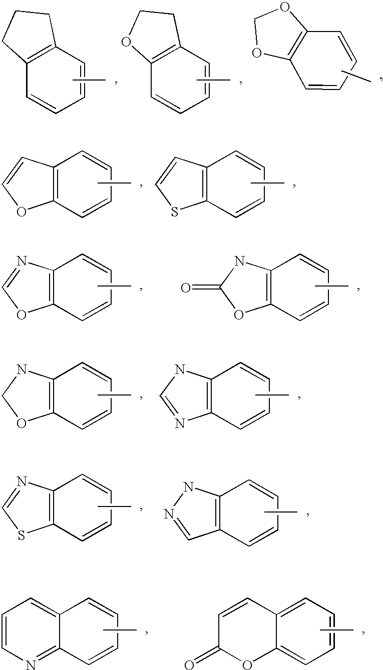

and may be optionally substituted through available carbon atoms with one or more substitutents, such as halo, haloalkyl, alkyl, haloalkyl, alkoxy, haloalkoxy, alkenyl, trifluoromethyl, trifluoromethoxy, alkynyl, cycloalkyl-alkyl, cycloheteroalkyl, cycloheteroalkylalkyl, aryl, heteroaryl, arylalkyl, aryloxy, aryloxyalkyl, arylalkoxy, alkoxycarbonyl, arylcarbonyl, arylalkenyl, aminocarbonylaryl, arylthio, arylsulfinyl, arylazo, heteroarylalkyl, heteroarylalkenyl, heteroarylheteroaryl, heteroaryloxy, hydroxy, nitro, cyano, amino, substituted amino wherein the amino includes 1 or 2 substituents (which are alkyl, aryl or any of the other aryl compounds mentioned in the definitions), thiol, alkylthio, arylthio, heteroarylthio, arylthioalkyl, alkoxyarylthio, alkylcarbonyl, arylcarbonyl, alkylaminocarbonyl, arylaminocarbonyl, alkoxycarbonyl, aminocarbonyl, alkylcarbonyloxy, arylcarbonyloxy, alkylcarbonylamino, arylcarbonylamino, arylsulfinyl, arylsulfinylalkyl, arylsulfonylamino or arylsulfonaminocarbonyl and/or any of the alkyl substituents set out herein.

The term heterocycle, hetero or heterocyclic ring, as used herein, represents an unsubstituted or substituted stable 5- to 7-membered monocyclic ring system which may be saturated or unsaturated, and which consists of carbon atoms and from one to four heteroatoms selected from N, O or S, and wherein the sulfur heteroatoms may optionally be oxidized.

As used herein, the term “carbocycle” refers to cyclic groups in which the ring portion is composed solely of carbon atoms.

The term “prodrug esters” as employed herein includes esters and carbonates formed by reacting one or more hydroxyls of compounds of formula I with alkyl, alkoxy, or aryl substituted acylating agents employing procedures known to those skilled in the art to generate acetates, pivalates, methylcarbonates, benzoates and the like.

Any compound that can be converted in vivo to provide the bioactive agent (i.e., the compound of formula I) is a prodrug within the scope and spirit of the invention.

Various forms of prodrugs are well known in the art. A comprehensive description of prodrugs and prodrug derivatives are described in:

a.) The Practice of Medicinal Chemistry, Camille G. Wermuth et al., Ch 31, (Academic Press, 1996);

b.) Design of Prodrugs, edited by H. Bundgaard, (Elsevier, 1985); and

c.) A Textbook of Drug Design and Development, P.

Krogsgaard-Larson and H. Bundgaard, eds. Ch 5, pgs 113-191 (Harwood Academic Publishers, 1991).

Said references are incorporated herein by reference.

An administration of a therapeutic agent of the invention includes administration of a therapeutically effective amount of the agent of the invention. The term “therapeutically effective amount” as used herein refers to an amount of a therapeutic agent to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The precise effective amount for a subject will depend upon the subject's size and health, the nature and extent of the condition being treated, recommendations of the treating physician, and the therapeutics or combination of therapeutics selected for administration. Thus, it is not useful to specify an exact effective amount in advance.

The term “other type of therapeutic agents” as employed herein includes, but is not limited to one or more antidiabetic agents (other than SGLT2 inhibitors of formula I), one or more anti-obesity agents, one or more anti-hypertensive agents, one or more anti-platelet agents, one or more anti-atherosclerotic agents and/or one or more lipid-lowering agents (including anti-atherosclerosis agents).

All stereoisomers of the compounds of the instant invention are contemplated, either in admixture or in pure or substantially pure form. The compounds of the present invention can have asymmetric centers at any of the carbon atoms including any one of the R substituents. Consequently, compounds of formula I can exist in enantiomeric or diastereomeric forms or in mixtures thereof. The processes for preparation can utilize racemates, enantiomers or diastereomers as starting materials. When diastereomeric or enantiomeric products are prepared, they can be separated by conventional methods for example, chromatographic or fractional crystallization.

The compounds of formula I of the invention can be prepared as shown in the following reaction schemes and description thereof, as well as relevant published literature procedures that may be used by one skilled in the art. Exemplary reagents and procedures for these reactions appear hereinafter in the working Examples.

Compounds of formula I of the invention can be prepared from compounds of formula II

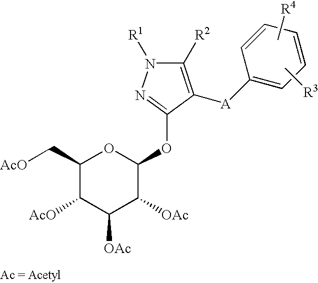

by treatment with a base such as LiOH or NaOH in a solvent such as 3:1 MeOH/H₂O or 3:2:1 MeOH/THF/H₂O.

Compounds of formula II can be prepared by reacting commercially available 2,3,4,6-tetra -O-acetyl-α-D-glucopyranosyl bromide III

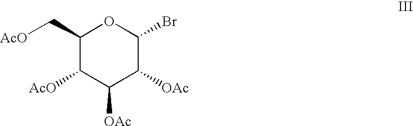

with compounds of formula IV

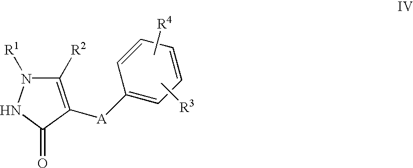

in the presence of Ag₂O in a solvent such as lutidine or quinoline or in the presence of silver triflate in a 5 solvent such as CH₂Cl₂ containing a base such as 2,6-di-t-butyl-4-methylpyridine.

Scheme I below illustrates a preferred means for preparing the compounds of this invention.

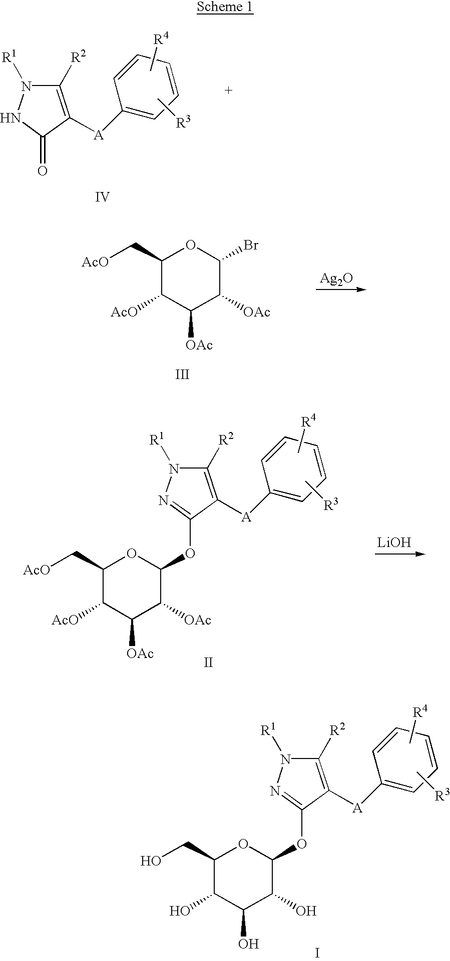

Compounds of formula IV where R¹ is H can be prepared from compounds of formula V

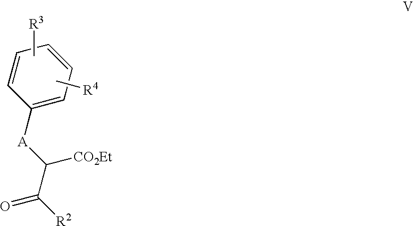

upon heating with hydrazine in a 3% MeOH/tolune solution containing HOAc as catalyst.

Compounds of formula V are readily prepared from compounds of formula VI

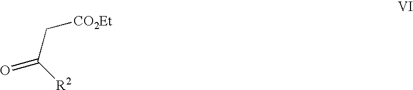

or by sequentially heating with NaH in a solvent such as DME followed by alkylation with a compound of formula VII

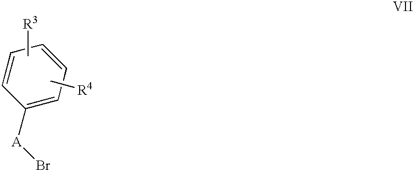

or by a variety of alternative conditions familiar to those skilled in the art.

Compounds of formula VI and formula VII are either commercially available or readily prepared by one skilled in the art.

Compounds of formula IV where R¹ is alkyl, alkenyl, or arylalkyl can be prepared from compounds of formula IV where R¹ is hydrogen by sequential treatment with a base such as n-BuLi in a solvent such as THF followed by either commercially available or readily accessible alkylating agents such as compounds of formula VII, where A is either CH₂, (CH₂)₂, or allyl.

### USE & UTILITY

A. Utilities

The compounds of the present invention possess activity as inhibitors of the sodium dependent glucose transporters found in the intestine and kidney of mammals. Preferably, the compounds of the invention are inhibitors of renal SGLT2 activity and therefore may be used in the treatment of diseases or disorders associated with SGLT2 activity.

Accordingly, the compounds of the present invention can be administered to mammals, preferably humans, for the treatment of a variety of conditions and disorders, including, but not limited to, treating or delaying the progression or onset of diabetes(including Type I and Type II, impaired glucose tolerance, insulin resistance, and diabetic complications, such as nephropathy, retinopathy, neuropathy and cataracts), hyperglycemia, hyperinsulinemia, hypercholesterolemia, elevated blood levels of free fatty acids or glycerol, hyperlipidemia, hypertriglyceridemia, obesity, wound healing, tissue ischemia, atherosclerosis and hypertension. The compounds of the present invention may also be utilized to increase the blood levels of high density lipoprotein (HDL).

In addition, the conditions, diseases, and maladies collectively referenced to as “Syndrome X” or Metabolic Syndrome as detailed in Johannsson J. Clin. Endocrinol. Metab., 82, 727-34 (1997), may be treated employing the compounds of the invention.

B. Combinations

The present invention includes within its scope pharmaceutical compositions comprising, as an active ingredient, a therapeutically effective amount of at least one of the compounds of formula I, alone or in combination with a pharmaceutical carrier or diluent. Optionally, compounds of the present invention can be used alone, in combination with other compounds of the invention, or in combination with one or more other therapeutic agent(s), e.g., an antidiabetic agent or other pharmaceutically active material.

The compounds of the present invention may employed in combination with other inhibitors of SGLT2 activity or other suitable therapeutic agents useful in the treatment of the aforementioned disorders including: anti-diabetic agents; anti-hyperglycemic agents; hypolipidemic/lipid lowering agents; anti-obesity agents; anti-hypertensive agents and appetite supressants.

Examples of suitable anti-diabetic agents for use in combination with the compounds of the present invention include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, gliclazide, chlorpropamide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, glycogen phosphorylase inhibitors, inhibitors of fatty acid binding protein (aP2), glucagon-like peptide-1 (GLP-1), and dipeptidyl peptidase IV (DPP4) inhibitors.

It is believed that the use of the compounds of formula I in combination with at least one or more other antidiabetic agent(s) provides antihyperglycemic results greater than that possible from each of these medicaments alone and greater than the combined additive anti-hyperglycemic effects produced by these medicaments.

Other suitable thiazolidinediones include Mitsubishi's MCC-555 (disclosed in U.S. Pat. No. 5,594,016), Glaxo-Welcome's GL-262570, englitazone (CP-68722, Pfizer) or darglitazone (CP-86325, Pfizer, isaglitazone (MIT/J&J), JTT-501 (JPNT/P&U), L-895645 (Merck), R-119702 (Sankyo/WL), NN-2344 (Dr. Reddy/NN), or YM-440 (Yamanouchi).

Suitable PPAR alpha/gamma dual agonists include AR-HO39242 (Astra/Zeneca), GW-409544 (Glaxo-Wellcome), KRP297 (Kyorin Merck) as well as those disclosed by Murakami et al, “A Novel Insulin Sensitizer Acts As a Coligand for Peroxisome Proliferation—Activated Receptor Alpha (PPAR alpha) and PPAR gamma. Effect on PPAR alpha Activation on Abnormal Lipid Metabolism in Liver of Zucker Fatty Rats”, Diabetes 47, 1841-1847 (1998), and in U.S. application Ser. No. 09/644,598, filed Sep. 18, 2000, the disclosure of which is incorporated herein by reference, employing dosages as set out therein, which compounds designated as preferred are preferred for use herein.

Suitable aP2 inhibitors include those disclosed in U.S. application Ser. No. 09/391,053, filed Sep. 7, 1999, and in U.S. application Ser. No. 09/519,079, filed Mar. 6, 2000, employing dosages as set out herein.

Suitable DPP4 inhibitors include those disclosed in WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) as disclosed by Hughes et al, Biochemistry, 38(36), 11597-11603, 1999, TSL-225 (tryptophyl-1,2,3,4-tetrahydroisoquinoline-3-carboxylic acid (disclosed by Yamada et al, Bioorg. & Med. Chem. Lett. 8 (1998) 1537-1540, 2-cyanopyrrolidides and 4- cyanopyrrolidides, as disclosed by Ashworth et al, Bioorg. & Med. Chem. Lett., Vol. 6, No. 22, pp 1163-1166 and 2745-2748 (1996) employing dosages as set out in the above references.

Other suitable meglitinides include nateglinide (Novartis) or KAD1229 (PF/Kissei).

Examples of suitable anti-hyperglycemic agents for use in combination with the compounds of the present invention include glucagon-like peptide-1 (GLP-1,) such as GLP-l(l-36) amide, GLP-1(7-36) amide, GLP-1(7-37) (as disclosed in U.S. Pat. No. 5,614,492 to Habener), as well as AC2993 (Amylen) and LY-315902 (Lilly).

Examples of suitable hypolipidemic/lipid lowering agents for use in combination with the compounds of the present invention include one or more MTP inhibitors, HMG CoA reductase inhibitors, squalene synthetase inhibitors, fibric acid derivatives, ACAT inhibitors, lipoxygenase inhibitors, cholesterol absorption inhibitors, ileal Na⁺/bile acid cotransporter inhibitors, upregulators of LDL receptor activity, bile acid sequestrants, cholesterol ester transfer protein inhibitors (e.g., CP-529414 (Pfizer)) and/or nicotinic acid and derivatives thereof.

MTP inhibitors which may be employed as described above include those disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440.

The HMG CoA reductase inhibitors which may be employed in combination with one or more compounds of formula I include mevastatin and related compounds, as disclosed in U.S. Pat. No. 3,983,140, lovastatin (mevinolin) and related compounds, as disclosed in U.S. Pat. No. 4,231,938, pravastatin and related compounds, such as disclosed in U.S. Pat. No. 4,346,227, simvastatin and related compounds, as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin, as disclosed in U.S. Pat. Nos. 5,006,530 and 5,177,080, atorvastatin, as disclosed in U.S. Pat. Nos. 4,681,893, 5,273,995, 5,385,929 and 5,686,104, atavastatin (Nissan/Sankyo's nisvastatin (NK-104)), as disclosed in U.S. Pat. No. 5,011,930, visastatin (Shionogi-Astra/Zeneca (ZD-4522)), as disclosed in U.S. Pat. No. 5,260,440, and related statin compounds disclosed in U.S. Pat. No. 5,753,675, pyrazole analogs of mevalonolactone derivatives, as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No.0142146 A2, and quinoline and pyridine derivatives, as disclosed in U.S. Pat. No. 5,506,219 and 5,691,322.

Preferred hypolipidemic agents are pravastatin, lovastatin, simvastatin, atorvastatin, fluvastatin, cerivastatin, atavastatin and ZD-4522.

In addition, phosphinic acid compounds useful in inhibiting HMG CoA reductase, such as those disclosed in GB 2205837, are suitable for use in combination with the compounds of the present invention.

The squalene synthetase inhibitors suitable for use herein include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinyl-methyl)phosphonates, as well as other known squalene synthetase inhibitors, for example, as disclosed in U.S. Pat. No. 4,871,721 and 4,924,024 and in Biller, S. A., Neuenschwander, K., Ponpipom, M. M., and Poulter, C. D., Current Pharmaceutical Design, 2, 1-40 (1996).

In addition, other squalene synthetase inhibitors suitable for use herein include the terpenoid pyrophosphates disclosed by P. Ortiz de Montellano et al, J. Med. Chem., 1977, 2-, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, Summary.

The fibric acid derivatives which may be employed in combination with one or more compounds of formula I include fenofibrate, gemfibrozil, clofibrate, bezafibrate, ciprofibrate, clinofibrate and the like, probucol, and related compounds, as disclosed in U.S. Pat. No. 3,674,836, probucol and gemfibrozil being preferred, bile acid sequestrants, such as cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives, such as disclosed in U.S. Pat. No. 4,759,923, quaternary amine poly(diallyldimethylammonium chloride) and ionenes, such as disclosed in U.S. Pat. No. 4,027,009, and other known serum cholesterol lowering agents.

The ACAT inhibitor which may be employed in combination with one or more compounds of formula I include those disclosed in Drugs of the Future 24, 9-15 (1999), (Avasimibe); “The ACAT inhibitor, C1-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62, or TS-962 (Taisho Pharmaceutical Co. Ltd).

The hypolipidemic agent may be an upregulator of LD2 receptor activity, such as MD-700 (Taisho Pharmaceutical Co. Ltd) and LY295427 (Eli Lilly).

Examples of suitable cholesterol absorption inhibitor for use in combination with the compounds of the invention include SCH48461 (Schering-Plough), as well as those disclosed in Atherosclerosis 115, 45-63 (1995) and J. Med. Chem. 41, 973 (1998).

Examples of suitable ileal Na⁺/bile acid cotransporter inhibitors for use in combination with the compounds of the invention include compounds as disclosed in Drugs of the Future, 24, 425-430 (1999).

The lipoxygenase inhibitors which may be employed in combination with one or more compounds of formula I include 15-lipoxygenase (15-LO) inhibitors, such as benzimidazole derivatives, as disclosed in WO 97/12615, 15-LO inhibitors, as disclosed in WO 97/12613, isothiazolones, as disclosed in WO 96/38144, and 15-LO inhibitors, as disclosed by Sendobry et al “Attenuation of diet-induced atherosclerosis in rabbits with a highly selective 15-lipoxygenase inhibitor lacking significant antioxidant properties”, Brit. J. Pharmacology (1997) 120, 1199-1206, and Cornicelli et al, “15-Lipoxygenase and its Inhibition: A Novel Therapeutic Target for Vascular Disease”, Current Pharmaceutical Design, 1999, 5, 11-20.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable anti-obesity agents for use in combination with the compounds of the present invention include a beta 3 adrenergic agonist, a lipase inhibitor, a serotonin (and dopamine) reuptake inhibitor, a thyroid receptor beta drug and/or an anorectic agent.

The beta 3 adrenergic agonists which may be optionally employed in combination with compounds of the present invention include AJ9677 (Takeda/Dainippon), L750355 (Merck), or CP331648 (Pfizer,) or other known beta 3 agonists, as disclosed in U.S. Pat. Nos. 5,541,204, 5,770,615, 5,491,134, 5,776,983 and 5,488,064, with AJ9677, L750,355 and CP331648 being preferred.

Examples of lipase inhibitors which may be optionally employed in combination with compounds of the present invention include orlistat or ATL-962 (Alizyme), with orlistat being preferred.

The serotonin (and dopoamine) reuptake inhibitor which may be optionally employed in combination with a compound of formula I may be sibutramine, topiramate (Johnson & Johnson) or axokine (Regeneron), with sibutramine and topiramate being preferred.

Examples of thyroid receptor beta compounds which may be optionally employed in combination with compounds of the present invention include thyroid receptor ligands, such as those disclosed in W097/21993 (U. Cal SF), W099/00353 (KaroBio) and GB98/284425 (KaroBio), with compounds of the KaroBio applications being preferred.

The anorectic agent which may be optionally employed in combination with compounds of the present invention include dexamphetamine, phentermine, phenylpropanolamine or mazindol, with dexamphetamine being preferred.

The aforementioned patents and patent applications are incorporated herein by reference.

The above other therapeutic agents, when employed in combination with the compounds of the present invention may be used, for example, in those amounts indicated in the Physician's Desk Reference, as in the patents set out above or as otherwise determined by one of ordinary skill in the art.

Where the compounds of the invention are utilized in combination with one or more other therapeutic agent(s), either concurrently or sequentially, the following combination ratios and dosage ranges are preferred:

Where the other antidiabetic agent is a biguanide, the compounds of formula I will be employed in a weight ratio to biguanide within the range from about 0.01:1 to about 100:1, preferably from about 0.1:1 to about 5:1.

The compounds of formula I will be employed in a weight ratio to the glucosidase inhibitor within the range from about 0.01:1 to about 100:1, preferably from about 0.5:1 to about 50:1.

The compounds of formula I will be employed in a weight ratio to the sulfonyl urea in the range from about 0.01:1 to about 100:1, preferably from about 0.2:1 to about 10:1.

The compounds of formula I will be employed in a weight ratio to the thiazolidinedione in an amount within the range from about 0.01:1 to about 100:1, preferably from about 0.2:1 to about 10:1.

Where present, the thiazolidinedione anti-diabetic agent may be employed in amounts within the range from about 0.01 to about 2000 mg/day which may be administered in single or divided doses one to four times per day.

Optionally, the sulfonyl urea and thiazolidinedione may be incorporated in a single tablet with the compounds of formula I in amounts of less than about 150 mg.

Where present, metformin or salt thereof may be employed in amounts within the range from about 500 to about 2000 mg per day which may be administered in single or divided doses one to four times daily.

Where present GLP-1 peptides may be administered in oral buccal formulations, by nasal administration or parenterally as described in U.S. Pat. Nos. 5,346,701 (TheraTech), 5,614,492 and 5,631,224 which are incorporated herein by reference.

The SGLT2 inhibitor of formula I will be employed in a weight ratio to the meglitinide, PPAR-gamma agonist, PPAR-alpha/gamma dual agonist, aP2 inhibitor or DPP4 inhibitor within the range from about 0.01:1 to about 100:1, preferably from about 0.2:1 to about 10:1.

The compounds of formula I of the invention will be generally be employed in a weight ratio to the hypolipidemic agent (were present), within the range from about 500:1 to about 1:500, preferably from about 100:1 to about 1:100.

For oral administration, a satisfactory result may be obtained employing the MTP inhibitor in an amount within the range of from about 0.01 mg/kg to about 500 mg and preferably from about 0.1 mg to about 100 mg, one to four times daily.

A preferred oral dosage form, such as tablets or capsules, will contain the MTP inhibitor in an amount of from about 1 to about 500 mg, preferably from about 2 to about 400 mg, and more preferably from about 5 to about 250 mg, one to four times daily.

For oral administration, a satisfactory result may be obtained employing an HMG CoA reductase inhibitor in an amount within the range of from about 1 to 2000 mg, and preferably from about 4 to about 200 mg.

A preferred oral dosage form, such as tablets or capsules, will contain the HMG CoA reductase inhibitor in an amount from about 0.1 to about 100 mg, preferably from about 5 to about 80 mg, and more preferably from about 10 to about 40 mg.

The squalene synthetase inhibitor may be employed in dosages in an amount within the range of from about 10 mg to about 2000 mg and preferably from about 25 mg to about 200 mg.

A preferred oral dosage form, such as tablets or capsules will contain the squalene synthetase inhibitor in an amount of from about 10 to about 500 mg, preferably from about 25 to about 200 mg.

The compounds of the formula I can be administered for any of the uses described herein by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories; in dosage unit formulations containing non-toxic, pharmaceutically acceptable vehicles or diluents.

In carrying out a preferred method of the invention for treating any of the diseases disclosed herein, such as diabetes and related diseases, a pharmaceutical composition will be employed containing one or more of the compounds of formula I, with or without other antidiabetic agent(s) and/or antihyperlipidemic agent(s) and/or other type therapeutic agents in association with a pharmaceutical vehicle or diluent. The pharmaceutical composition can be formulated employing conventional solid or liquid vehicles or diluents and pharmaceutical additives of a type appropriate to the mode of desired administration, such as pharmaceutically acceptable carriers, excipients, binders and the like. The compounds can be administered to mammalian species including humans, monkeys, dogs, etc. by an oral route, for example, in the form of tablets, capsules, beads, granules or powders, or they can be administered by a parenteral route in the form of injectable preparations, or they can be administered intranasally or in transdermal patches. Typical solid formulations will contain from about 10 to about 500 mg of a compound of formula I. The dose for adults is preferably between 10 and 2,000 mg per day, which can be administered in a single dose or in the form of individual doses from 1-4 times per day.

A typical injectable preparation may be produced by aseptically placing 250 mg of compounds of formula I into a vial, aseptically freeze-drying and sealing. For use, the contents of the vial are mixed with 2 mL of physiological saline, to produce an injectable preparation.

It will be understood that the specific dose level and frequency of dosage for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition.

### Assay for SGLT2 Activity

The mRNA sequence for human SGLT2 (GenBank #M95549) was cloned by reverse-transcription and amplification from human kidney mRNA, using standard molecular biology techniques. The cDNA sequence was stably transfected into CHO cells, and clones were assayed for SGLT2 activity essentially as described in Ryan M J, Johnson G, Kirk J, Fuerstenberg S M, Zager R A, Torok-Storb B, “HK-2: an immortalized proximal tubule epithelial cell line from normal adult human kidney”, Kidney International 45: 48-57 (1994) (hereinafter “Ryan et al.”) Evaluation of inhibition of SGLT2 activity in a clonally selected cell line was performed essentially as described in Ryan et al., with the following modifications. Cells were grown in 96-well plates for 2-4 days to 75,000 or 30,000 cells per well in F-12 nutrient mixture (Ham's F-12), 10% fetal bovine serum, 300 ug/ml Geneticin and penicillin-streptomycin. At confluence, cells were washed twice with 10 mM Hepes/Tris, pH 7.4, 137 mM N-methyl-D-glucamine, 5.4 mM KCl, 2.8 mM CaCd₂, 1.2 mM MgSO₄. Cells then were incubated with 10 μM [¹⁴C]AMG, and 10 μM inhibitor (final DMSO=0.5%) in 10 mM Hepes/Tris, pH 7.4, 137 mM NaCl, 5.4 mM KCl, 2.8 mM CaCl₂, 1.2 mM MgSO₄ at 37° C. for 1.5 hr. Uptake assays were quenched once with ice cold PBS containing 0.5 mM phlorizin, and cells were then lysed with 0.1% NaOH. After addition of MicroScint scintillation fluid, the cells were allowed to shake for 1 hour, and then [¹⁴C]AMG was quantitated on a TopCount scintillation counter. Controls were performed with and without NaCl. For determination of EC₅₀ values, 10 inhibitor concentrations were used over 2 log intervals in the appropriate response range, and triplicate plates were averaged across plates.

The following working Examples serve to better illustrate, but not limit, some of the preferred embodiments of the present invention. All temperatures are expressed in degrees Centigrade unless otherwise indicated.

### EXAMPLE 1

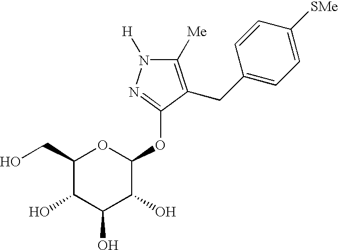

### Compound 1a

### Ethyl 2-(4methylthiophenylmethyl)-3-oxo-butanoate

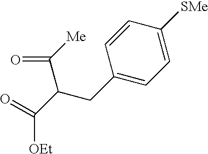

To a stirred 4° C. suspension of 60% NaH/mineral oil (0.43 g, 10 mmol)under argon in 5 mL THF was added ethyl acetoacetate (1.4 g, 10 mmol) without solvent at a rate such that H₂ gas evolution remained under control. Upon cessation of gas evolution, para methylthiobenzyl bromide (2.7 g, 10 mmol) was added. The resulting solution was refluxed for 20 hr whereupon, after cooling to 4° C., the reaction was quenched with 1N HCl prior to 3 EtOAc extracts. The organic fractions were washed once with H₂O and brine prior to drying over Na₂SO₄. After removal of the solvent the residue was chromatographed on silica gel. The desired benzylated ketoester (15 g) was eluted as a yellow oil with 15% EtOAc/hexane.

### Compound 1b

### 1,2-dihydro-4-[[4-methylthiophenyl]methyl]-5-methyl-3H-pyrazol-3-one

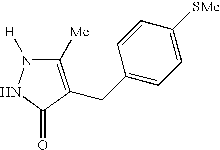

A mixture of ethyl 2-(4-methylthiophenylmethyl)-3-oxo-butanoate (Compound la),(200 mg, 0.75 mmol) and anhydrous hydrazine (48 mg, 1.5 mmol) in 15 mL of toluene was refluxed for 15 hr. After removal of the solvent, the residue was chromatographed on silica gel using 10% EtOAc/hexane to elute the desired 1,2-dihydro-4-[[4-methylthiophenyl]methyl]-5-methyl-3H-pyrazol-3-one (100 mg)

### Compound 1c

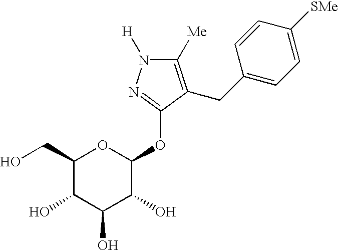

Following addition of 4 mL of lutidine to a mixture of 1,2-dihydro-4-[[4-methylthiophenyl]methyl]-5-methyl-3H-pyrazol-3-one(80 mg, 0.34 mmol) (Compound 1b), Ag₂O (110 mg, 0.5 mmol) and 2,3,4,6-tetra-O-acetyl-α-D-glucopyranosyl bromide (170 mg, 0.39 mmol), the suspension was stirred at 20° C. for 3 days, whereupon HPLC analysis showed no starting pyrazolone remained. The reaction was diluted with 50 mL of CH₂Cl₂ and filtered through celite. The volatiles were removed using a rotary evaporator; toluene was added and removed under vaccumn to drive out the residual lutidine. Upon dissolution of the residue in 5 mL MeOH followed by addition of 0.5 mL of 1N NaOH, the reaction was stirred for 75 minutes before quenching with 1M AcOH/MeOH. After adjustment of the pH to 7.5, the volatiles were removed using a rotary evaporator. The residue was purified by preparative HPLC employing MeOH/H₂O gradient elution from a YMC reverse phase column. Subsequent concentration and lyophilization yielded the title glucoside.

¹HNMR (CD₃OD, 400 MHz) : δ2.06 (s, 3H), 2.42 (s, 3H), 3.30-3.42 (m, 4H), 3.64-3.75 (m, 3H), 3.84 (d, 1H), 5.05 (d, 1H), 7.15 (s, 4H). HPLC retention time: 5.7 min, Zorbax C-18 4.6×75mm, 2.5 mL/min, detection at-220 nm, 8 min gradient O-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd for C₁₈H₂₄N₂O₆S LC/MS (M+H) 397

### EXAMPLE 2

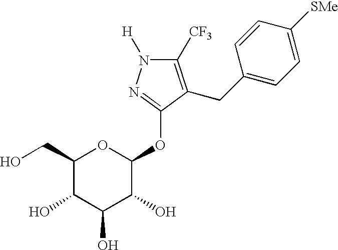

### Compound 2a

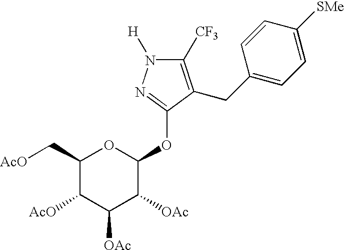

To a stirred 20° C. solution of 1,2-dihydro-4-[[4-methylthiophenyl]methyl]-5-trifluoromethyl-3H-pyrazol-3-one(88 mg, 0.3 mmol) which was prepared according to the procedure found in K. L. Kees et. al., J. Med. Chem., 1996, 39, 3920-3928, incorporated herein by reference, in 0.6 mL of quinoline was sequentially added Ag₂O (36 mg, 0.16 mmol) and 2,3,4,6-tetra-O-acetyl-α-D-glucopyranosyl bromide (141 mg, 0.32 mmol). After 4 hr, 11% of starting pyrazolone remained as determined by HPLC. An additional 20% of Ag₂O and pyranosyl bromide was added and the reaction continued for another 10 hr. An additional 0.4 mL of quinoline was required to dilute the suspension to maintain stirring. Upon completion of the reaction, the suspension was diluted with CH₂Cl₂ prior to filtration through celite. The filtrate was washed four times with 0.5N HCl, once H₂O, and once with brine prior to drying over Na₂SO₄. The residue, after removal of the volatiles using a rotary evaporator, was purified by silica gel chromatography. After elution of non polar impurities with 2:1 hexane/EtOAc, 3:2 hexane/EtOAc eluted the desired tetraacetylated glucoside (160 mg).

### Compound 2b:

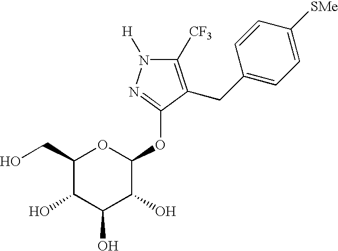

To a stirred solution of the tetraacetylated glucoside of Compound la (160 mg, 0.255 mmol) in 4 mL of 1:2:3 H₂O/THF/MeOH was added LiOH·H₂0 (50 mg, 1.25 mmol). After 8 hr, when the reaction was complete as determined by HPLC, the solution was neutralized with 1 N HCl before removal of the volatiles using a rotary evaporator. The residue was purified by preparative HPLC by MeOH/H₂O gradient elution from a YMC reverse phase column. Subsequent concentration and lyophilization yielded 110 mg of the title glucoside which was isolated as a white lyophilate.

¹HNMR (CD₃OD, 400 MHz): δ2.41 (s, 3H), 3.29-3.40 (m, 4H), 3.69-3.75 (dd, 1H), 3.83-3.86 (m, 3H), 4.98 (d, 1H), 7.13 (s, 4H). ¹³CNMR (CD₃OD, 100 MHz): δ16.18, 27.27, 62.43, 71.1, 74.75, 77.90, 78.49, 103.9, 128.07, 129.82, 137.3, 138.46. HPLC retention time: 6.9 min, Zorbax C-18 4.6×75mm, 2.5 mL/min, detection at 220 nm, 8 min gradient 0-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd for C₁₈H₂₁F₃N₂O₆S LC/MS (M+Na) 473

### EXAMPLE 3

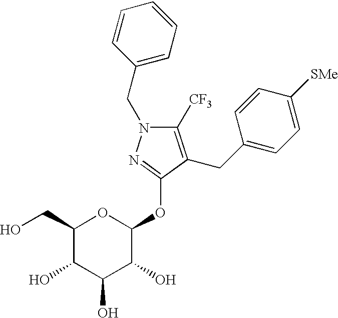

### Compound 3a:

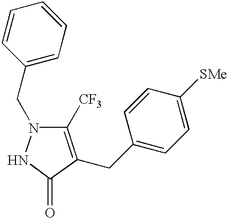

To a stirred 1 mL THF solution of 1,2-dihydro-4-[[4-methylthiophenyl]methyl]-5-trifluoromethyl-3H-pyrazol-3one(1.24 g, 4.3 mmol), prepared according to the procedure of K. L. Kees et. al., J. Med. Chem., 1996, 39, 3920-3928,incorporated herein by reference, at −78° C. under Ar, was added 16 mL of 1.6M nBuLi/hexane. After 15 minutes, benzyl bromide (6 g, 35 mmol) was added. The resulting solution after warming to 20° C., was stirred for 48 hr before being quenched with NH₄Cl/H₂O. The mixture was extracted twice with EtOAc. The organic fractions were washed once with H₂O and brine prior drying over Na₂SO₄. After removal of the solvent, the residue was chromatographed on silica gel. Impure benzylated pyrazolone was eluted with 10% EtOAc/hexane. Purification was subsequently achieved by preparative HPLC to yield 50 mg of N1-benzyl-1,2-dihydro-4-[[4-methylthiophenyl]methyl]-5-trifluoromethyl-3H-pyrazol-3-one.

### Compound 3b:

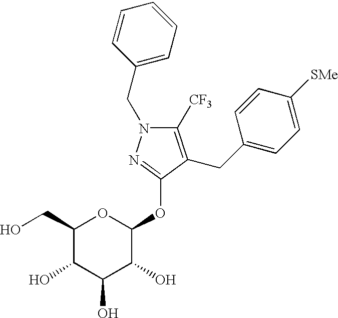

To a stirred 20° solution of N1-benzyl-1,2-dihydro-4-[[4-methylthiophenyl]methyl]-5-trifluoromethyl-3H-pyrazol-3-one (Compound 3a), (50 mg, 0.132 mmol) in 0.5 mL of lutidine was sequentially added Ag₂O (46 mg, 0.2 mmol) and 2,3,4,6-tetra-O-acetyl-α-D-glucopyranosyl bromide (108 mg, 0.265 mmol). The mixture was stirred for 3 hr before removal of the volatiles. After suspension of the residue in 0.5 mL of MeOH, 0.5 mL of 1N NaOH was added. After 3 hr, the pH was adjusted using 1N HCl to 7 prior to filtration of the salts and removal of the volatiles. Purification of the crude product by preparative HPLC yielded 20 mg of the title benzylated glucoside.

¹HNMR (CD₃OD, 400 MHz) : δ2.33 (s, 3H), 3.30-3.42 (m, 4H), 3.58-3.62 (dd, 1H), 3.70-3.72 (d, 1H), 3.74 (s, 2H), 5.22 (s, 2H), 5.27 (d, 1H), 7.01-7.06 (m, 6H),7.19-7.27 (m, 3H). HPLC retention time: 8.1 min, Zorbax C-18 4.6×75mm, 2.5 mL/min, detection at 220 nm, 8 min gradient 0-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd for C₂₅H₂₇F₃N₂O₆S LC/MS (M+H) 541

### EXAMPLE 4

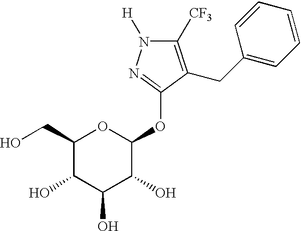

Following the procedures described in Example 2, 1,2-dihydro-4-[[phenyl]methyl]-5-trifluoromethyl-3H-pyrazol-3-one was converted to the title glucoside.

HPLC retention time: 3.3 min, YMC C-18 4.6×75mm, 2.5 mL/min, detection at 220 nm, 4 min gradient O-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd for C₁₇H₁₉F₃N₂O₆ LC/MS (M+Na) 427

### EXAMPLE 5

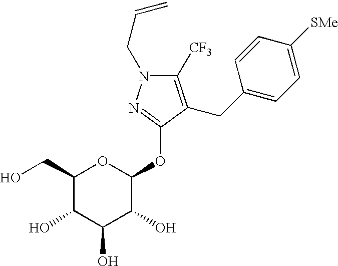

Following the procedures described in Example 3, 1,2-dihydro-4-[[4-methylthiophenyl]methyl]-5-trifluoromethyl-3H-pyrazol-3-one was alkylated with allyl bromide, rather than benzyl bromide, and using the technique described in Example 3, subsequently converted to the title glucoside. HPLC retention time: 7.5 min, Zorbax C-18 4.6×75mm, 2.5 mL/min, detection at 220 nm, 8 min gradient 0-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd for C₂₁H₂₅F₃N₂O₆S LC/MS (M+H) 491

### EXAMPLE 6

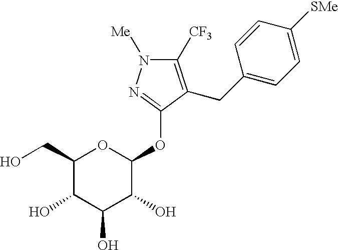

Following the procedures described in-Example 3, 1,2-dihydro-4-[[4-methylthiophenyl]methyl]-5-trifluoromethyl-3H-pyrazol-3-one was alkylated with methyl iodide and using the technique described in Example 3, subsequently converted to the title glucoside. HPLC retention time: 7.1 min, Zorbax C-18 4.6×75mm, 2.5 mL/min, detection at 220 nm, 8 min gradient O-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd for C₁₉H₂₃F₃N₂O₆S LC/MS (M+Na) 487

### EXAMPLE 7

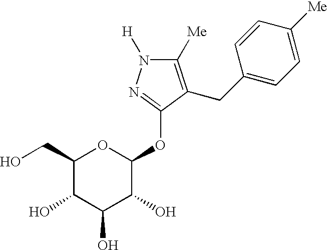

Following the procedures described in Example 1, ethyl acetoacetate and para methylbenzyl bromide were condensed to form ethyl 2-(4-methylphenylmethyl)-3-oxo-butanoate which was converted to 1,2-dihydro-4-[[4-methylphenyl]methyl]-5-methyl-3H-pyrazol-3-one. The latter was subsequently converted to the title glucoside using the procedure as described in Example 1c. HPLC retention time: 5.7 min, Zorbax C-18 4.6×75mm, 2.5 mL/min, detection at 220 nm, 8 min gradient O-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd for C₁₈H₂₄N₂O₆ LC/MS (M+Na) 387

### EXAMPLE 8

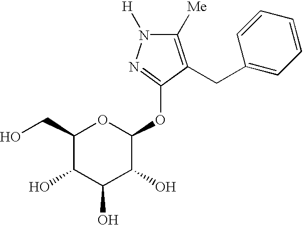

Following the procedures described in Example 1, ethyl 2-(4-phenylmethyl)-3-oxo-butanoate was prepared by condensation of ethyl acetoacetate and benzyl bromide and converted to 1,2-dihydro-4-[[4-phenyl]methyl]-5-methyl-3H-pyrazol-3-one which was subsequently converted to the title glucoside using the procedure as described in Example 1c. HPLC retention time: 2.7 min, YMC C-18 4.6×75mm, 2.5 mL/min, detection at 220 nm, 4 min gradient 0-100% solvent B hold 3 min at 100% solvent B. Solvent A: 10% MeOH/H₂O+0.2% H₃PO₄. Solvent B: 90% MeOH/H₂O+0.2% H₃PO₄. Anal Calcd. for C₁₇H₂₂N₂O₆ LC/MS (M+Na) 373

What is claimed:

1. A compound of the formula I 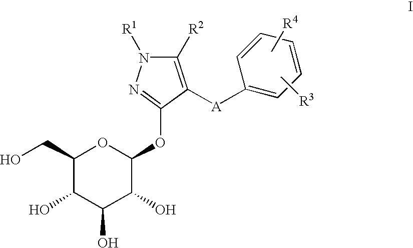
wherein;
A is CH₂ or (CH₂)₂;
R¹ is hydrogen, arylalkyl, alkenyl or alkyl;
R² is alkyl or perfluoroalkyl;
R³ and R⁴ are independently hydrogen, OH, OR⁵, OAryl, OCH₂Aryl, alkyl, cycloalkyl, CF₃, —OCHF₂, -3,4-(OCH₂O), —OCF₃, halogen, —CN, —CO₂R^{5a}, —CO₂H, —COR⁶, —CH(OH)R^{6a}, —CH(OR_{5b})R^{6b}, —CONR^{6c}R^{6d}, —NHCOR^{5c}, —NHSO₂R^{5d}, —NHSO₂Aryl, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂, or R³ and R⁴ together with the carbons to which they are attached form an annelated five, six or seven membered carbocycle or heterocycle which may contain 1 to 4 heteroatoms in the ring which are N, O, S, SO, and/or SO₂;
R⁵, R^{5a}, R^{5b}, R^{5c}, R^{5d}, R^{5e}, R^{5f}, and R^{5g}, are independently alkyl; and
R⁶, R^{6a}, R^{6b}, R^{6c} and R^{6d} are independently hydrogen, alkyl, aryl, arylalkyl or cycloalkyl, or R^{6c} and R^{6d} together with the nitrogen to which they are attached form an annelated five, six or seven membered heterocycle which may contain 1 to 4 heteroatoms in the ring which are N, O, S, SO, and/or SO₂, or a prodrug ester, pharmaceutically acceptable salt or stereoisomer thereof.

2. The compound as defined in claim 1 wherein A is CH₂;
R¹ is hydrogen or benzyl; and
R³ and R⁴ are independently hydrogen, OR⁵, OAryl, OCH₂Aryl, -3,4-(OCH₂O), alkyl, cycloalkyl, CF₃, —OCHF₂, —OCF₃, halogen, —CO₂R^{5a}, —COR⁶, —CH(OH)R^{6a}, —CH(OR^{5b})R^{6b}, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂, or R³ and R⁴ together with the carbons to which they are attached form an annelated five, six or seven membered carbocycle.

3. The compound as defined in claim 1 having the structure 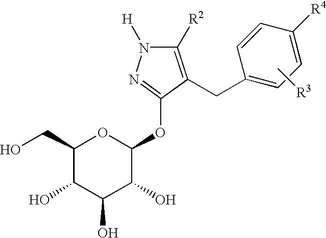
wherein
R³ is hydrogen; and
R⁴ is hydrogen, OR⁵, OAryl, OCH₂Aryl, -3,4-(OCH₂O), alkyl, cycloalkyl, CF₃, —OCHF₂, —OCF₃, halogen, —CO₂R^{5a}, —COR⁶, —CH(OH)R^{6a}, —CH(OR^{5b})R^{6b}, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂.

4. The compound as defined in claim 1 having the structure

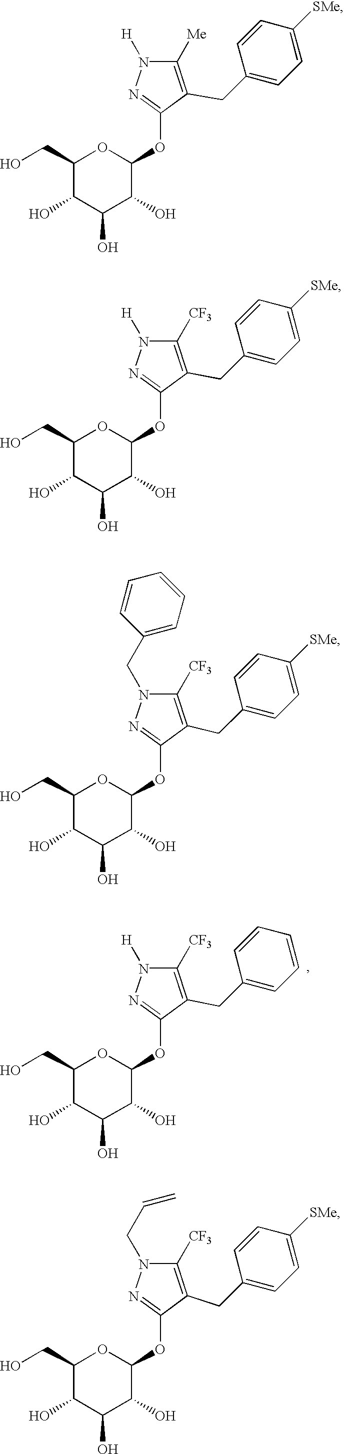

5. A pharmaceutical composition comprising a compound as defined in claim 1 and a pharmaceutically acceptable carrier therefor.

6. A pharmaceutical combination comprising a compound as defined in claim 1 and at least one therapeutic agent selected from the group consisting of an antidiabetic agent, an anti-obesity agent, a anti-hypertensive agent, an anti-atherosclerotic agent and a lipid-lowering agent.

7. The pharmaceutical combination as defined in claim 6 comprising the compound as defined in claim 1 and an antidiabetic agent.

8. The combination as defined in claim 7 wherein the antidiabetic agent is at least one agent selected from the group consisting of a biguanide, a sulfonyl urea, a glucosidase inhibitor, a PPAR γ agonist, a PPAR α/γ dual agonist, an aP2 inhibitor, a DP4 inhibitor, an insulin sensitizer, a glucagon-like peptide-1 (GLP-1), insulin and a meglitinide.

9. The combination as defined in claim 8 wherein the antidiabetic agent is at least one agent selected from the group consisting of metformin, glyburide, glimepiride, glipyride, glipizide, chlorpropamide, gliclazide, acarbose, miglitol, pioglitazone, troglitazone, rosiglitazone, insulin, G1-262570, isaglitazone, JTT-501, NN-2344, L895645, YM-440, R-119702, AJ9677, repaglinide, nateglinide, KAD1129, AR-H039242, GW-409544, KRP297, AC2993, LY315902, and NVP-DPP-728A.

10. The combination as defined in claim 7 wherein the compound is present in a weight ratio to the antidiabetic agent in the range of about 0.01 to about 300:1.

11. The combination as defined in claim 6 wherein the anti-obesity agent is at least one agent selected from the group consisting of a beta 3 adrenergic agonist, a lipase inhibitor, a serotonin (and dopamine) reuptake inhibitor, a thyroid receptor beta compound, and an anorectic agent.

12. The combination as defined in claim 11 wherein the anti-obesity agent is at least one agent selected from the group consisting of orlistat, ATL-962, AJ9677, L750355, CP331648, sibutramine, topiramate, axokine, dexamphetamine, phentermine, phenylpropanolamine, and mazindol.

13. The combination as defined in claim 6 wherein the lipid lowering agent is at least one agent selected from the group consisting of an MTP inhibitor, cholesterol ester transfer protein, an HMG CoA reductase inhibitor, a squalene synthetase inhibitor, a fibric acid derivative, an upregulator of LDL receptor activity, a lipoxygenase inhibitor, or an ACAT inhibitor.

14. The combination as defined in claim 13 wherein the lipid lowering agent is at least one agent selected from the group consisting of pravastatin, lovastatin, simvastatin, atorvastatin, cerivastatin, fluvastatin, nisvastatin, visastatin, fenofibrate, gemfibrozil, clofibrate, avasimibe, TS-962, MD-700, CP-529414, and/or LY295427.

15. The combination as defined in claim 13 wherein the compound as defined in claim 1 is present in a weight ratio to the lipid-lowering agent in the range of about 0.01 to about 100:1.

16. A method for treating or delaying the progression or onset of diabetes, diabetic retinopathy, diabetic neuropathy, diabetic nephropathy, wound healing, insulin resistance, hyperglycemia, hyperinsulinemia, Syndrome X, diabetic complications, elevated blood levels of free fatty acids or glycerol, hyperlipidemia, obesity, hypertriglyceridemia, atherosclerosis or hypertension, which comprises administering to a mammalian species in need of treatment a therapeutically effective amount of a compound as defined in claim 1.

17. A method according to claim 16 further comprising administering, concurrently or sequentially, a therapeutically effective amount of at least one additional therapeutic agent selected from the group consisting of an antidiabetic agent, an anti-obesity agent, a anti-hypertensive agent, an anti-atherosclerotic agent and a lipid-lowering agent.

18. A method for increasing the blood levels of high density lipoprotein (HDL) comprising administering a therapeutically effective amount of a compound as defined in claim 1.
